(19) United States
(12) Patent Application Publication
(10) Pub. No.: US 2005/0240157 A1
(43) Pub. Date: Oct. 27, 2005

(54) Multi-stage drug administration device and method

(76) Inventors: Shai Amisar, Tel Aviv (IL); Ronen Radomski, Haifa (IL); Baruch Segal, Hod-Hasharon (IL)

Correspondence Address: Edward Langer;c/o Shiboleth, Yisraeli, Roberts, Zisman & Co., 60th Floor, 350 Fifth Avenue, New York, NY, 10118, US

(21) Appl. No.: 11/109,862

(22) Filed: Apr. 20, 2005

(30) Foreign Application Priority Data
Apr. 21, 2004 (IL) 161552

(51) Int. Cl. A61M 5/178

(52) U.S. Cl. 604/185; 604/191

(57) ABSTRACT

A multi-stage drug administration device which, after connection to a standard IV port in the arm of a patient, administers the entire batch of drugs required by a patient automatically. The inventive device operates without any required stages of flushing, requiring no further supervision by a medical operative, thereby saving manpower and enabling full mobility of the patient while the drugs are being administered.

### FIELD OF THE INVENTION

The present invention relates to an apparatus and method for dispensing therapeutic drugs via infusion.

### BACKGROUND OF THE INVENTION

In the field of drug administration, there are many cases in which it is required to administer to a patient several types of drugs in series, via a standard IV port located in a patient's arm or other accessible vein. This normally requires a number of steps, including the following: first, flushing the IV port with a saline solution by means of a syringe; second, connecting to the IV port an infusion bag containing a first drug; third, after the entire amount of drug has been administered, removing the infusion bag and repeating the step of flushing; fourth, connecting to the IV port an infusion bag containing a second drug. This process is repeated for as many drugs as are required to be administered.

A disadvantage with the above-described process, is that it is labor intensive, requiring a medical operative to perform all the stages of connecting and disconnecting the syringes and infusion bags, and that it also substantially immobilizes the patient, until the entire batch of drugs has been fully administered.

Therefore, it would be desirable to provide an apparatus which simplifies the administration to a patient of a batch of drugs.

### SUMMARY OF THE INVENTION

Accordingly, it is a principal object of the present invention to overcome the disadvantages of prior art drug administration procedures and provide a multi-stage drug administration device which, after connection to a standard IV port in the arm of a patient, administers the entire batch of drugs required by a patient automatically. The inventive device operates without any required stages of flushing, requiring no further supervision by a medical operative, thereby saving manpower and enabling full mobility of the patient while the drugs are being administered.

There is thus provided, in accordance with a preferred embodiment of the invention, a multi-stage drug administration device for administering a sequence of pharmacological agents to a patient, which includes:

a generally cylindrical housing having distal and proximal ends, having formed at the distal end an outlet port for connection to an IV port; and

at least first and second compressible, mutually sealed chambers located within the housing between the distal and proximal ends, for containing therebetween in mutual isolation a corresponding plurality of pharmacological agents for sequential administration to a patient, wherein a rupture susceptible portion of a common dividing wall between adjacent chambers is adapted to rupture in the presence of at least a critical pressure differential thereacross,

wherein upon exposure to a predetermined source of pressure associated with the proximal end of the housing, the plurality of sealed chambers becomes compressed towards the distal end so as to cause an outflow of the pharmacological agent in the first chamber through the outlet port,

and wherein, at the time of emptying of the first chamber, the pressure differential across the rupture susceptible portion of the common dividing wall reaches the critical pressure differential causing rupture thereof, and a consequent outflow of the pharmacological agent contained in the second chamber through the outlet port.

Additionally in accordance with a preferred embodiment of the present invention, the common dividing wall is a disk-shaped piston spaced apart along the length of the cylinder, each piston being circumferentially sealed with an interior wall surface of the cylinder, thereby to form the plurality of chambers, each piston being adapted to rupture in the presence of the critical pressure differential.

Additionally in accordance with a preferred embodiment of the present invention, the device also includes puncturing apparatus located at the distal end, for puncturing a piston proximal thereto, in the presence of the critical pressure differential.

Additionally in accordance with a preferred embodiment of the present invention, each piston has formed thereon additional puncturing apparatus for puncturing an adjacent piston when brought into proximity therewith.

Additionally in accordance with a preferred embodiment of the present invention, each chamber includes a volume of a preselected pharmacological agent, contained within an impervious membrane.

Additionally in accordance with a preferred embodiment of the present invention, the impervious membrane is a flexible membrane.

In accordance with an alternative embodiment of the present invention, there is provided a multi-stage drug administration system for administering a sequence of pharmacological agents to a patient, which includes:

a generally cylindrical housing having distal and proximal ends, having formed at the distal end an outlet port for connection to an IV port;

at least first and second compressible, mutually sealed chambers located within the housing between the distal and proximal ends, for containing therebetween in mutual isolation a corresponding plurality of pharmacological agents for sequential administration to a patient, wherein a rupture susceptible portion of a common dividing wall between adjacent chambers is adapted to rupture in the presence of at least a critical pressure differential thereacross; and

a source of pressure associated with the proximal end of the housing,

wherein upon exposure to the pressure source, the plurality of sealed chambers becomes compressed towards the distal end so as to cause an outflow of the pharmacological agent in the first chamber through the outlet port,

and wherein, at the time of emptying of the first chamber, the pressure differential across the rupture susceptible portion of the common dividing wall reaches the critical pressure differential causing rupture thereof, and a consequent outflow of the pharmacological agent contained in the second chamber through the outlet port.

In accordance with yet a further embodiment of the present invention, there is provided a multi-stage drug administration system for administering a sequence of pharmacological agents to a patient, which includes:
a plurality of mutually connectable sealed modules, each containing a preselected pharmacological agent, arranged in a connected stack which includes a proximal end module and a distal end module;
a dispensing module connected to the distal end module;
and a pressure module, for connecting a source of pressure to the proximal end module.

The multi-stage drug administration system may further comprise a housing and a drive element, wherein said housing is adapted for disposing the modules and the drive element therewithin such that the drive element is in contact with the proximal module, and for attaching the dispensing module to a distal end thereof and the pressure module to a proximal end thereof.

The housing of the multi-stage drug administration system may comprise one or more narrow openings through which portions of stopper pins may be externally introduced into the interior of the housing such that drug administration is halted whenever the drive element reaches the location of a stopper pin which blocks further advancement thereof.

Additional features and advantages of the invention will become apparent from the following drawings and description.

### BRIEF DESCRIPTION OF THE DRAWINGS

For a better understanding of the invention with regard to the embodiments thereof, reference is made to the accompanying drawings, in which like numerals designate corresponding sections or elements throughout, and in which:

FIGS. 1A-1C are schematic representations of a multi-stage drug administration device, constructed and operative in accordance with a first embodiment of the invention;

FIG. 2 is a variation of the embodiment of FIGS. 1A-1C;

FIG. 3 is yet a further variation of the embodiment of FIGS. 1A-1C;

FIGS. 4A-4C illustrate a device which is generally similar to that depicted in FIGS. 1A-1C, but having a modular construction, in accordance with an alternative embodiment of the present invention;

FIGS. 5A-5E illustrate a quasi-modular drug administration device, constructed in accordance with yet a further embodiment of the present invention;

FIGS. 6A-6D illustrate a quasi-modular drug administration device constructed in accordance with an additional embodiment of the present invention;

FIG. 6E illustrates a collapsible module employed in the embodiment of FIGS. 6A-6D, in a compressed configuration, prior to use;

FIG. 6F illustrates the collapsible module of FIG. 6E, in an expanded configuration;

FIGS. 7A-7B illustrate a variation of the device of FIGS. 6A-6E; and

FIGS. 8A-8D illustrate a quasi-modular drug administration device wherein the drug administration may be halted at predetermined stages of administration.

### DETAILED DESCRIPTION OF THE PREFERRED EMBODIMENTS

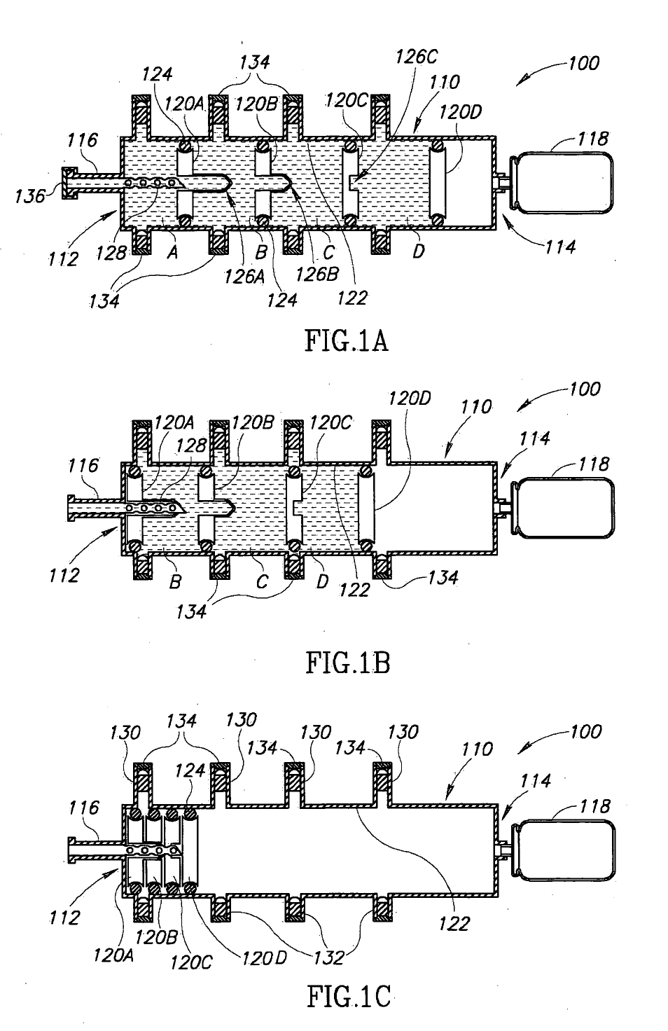

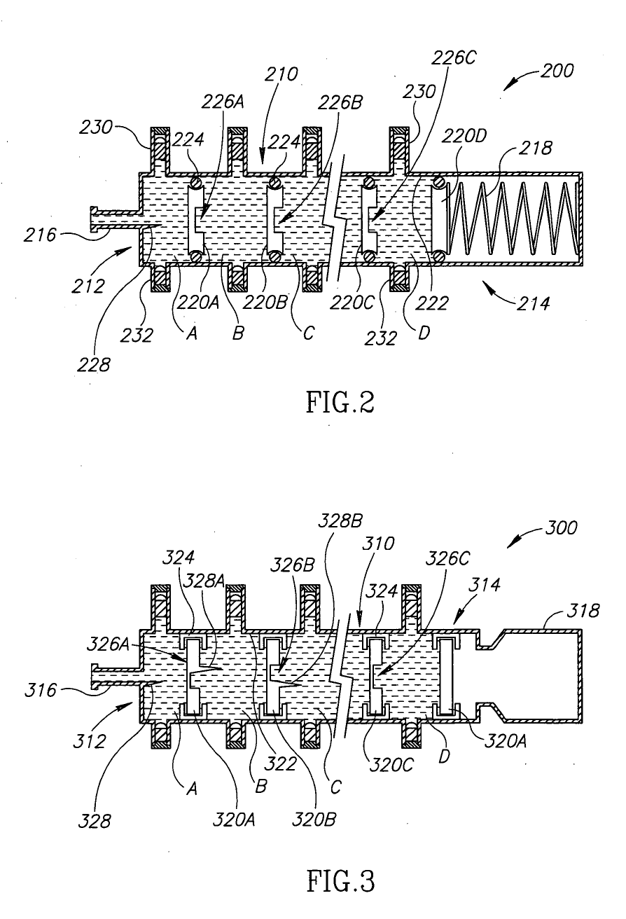

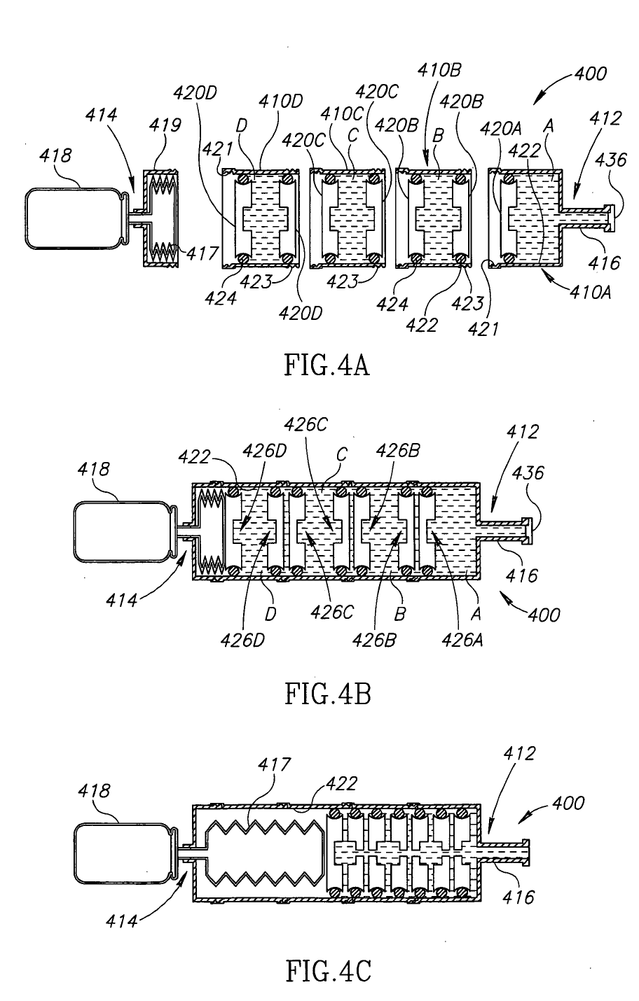

Referring now to the drawings generally, there is provided a multi-stage drug administration device for administering a sequence of pharmacological agents to a patient. While each different embodiment shown and described hereinbelow has specific features special to that embodiment, various features of the present invention may be found in all embodiments, either in an identical form, or in a form whose function or purpose is equivalent to those in other embodiments; these features are thus are not described separately with regard to each embodiment, except as may be required for an understanding of that embodiment, and are denoted in the drawings by reference numerals which are the same throughout, except for the addition of a prefix indicating the Figure numbers. Thus, for example, the multi-stage drug administration device of the invention is referenced 100 in FIGS. 1A-1C, 200 in FIG. 2, and 300 in FIG. 3, and so on. Furthermore, features common to more than one embodiment of the invention will normally be described with respect to the first embodiment in which they occur only, unless otherwise required for an understanding of the later embodiment.

Referring now to FIGS. 1A-1C, device 100 is formed of a generally cylindrical housing, referenced 110, having distal and proximal ends, respectively referenced 112 and 114. An outlet port 116, adapted for connection to an IV port (not shown), is provided at distal end 112, and a driver 118 is arranged in association with proximal end 114.

In the present embodiment, it is seen in FIG. 1A that in an initial position, prior to use, a plurality of compressible, mutually sealed chambers a, b, c, and d is contained within the housing 110 between the distal and proximal ends 112 and 114, each containing in mutual isolation a plurality of pharmacological agents for sequential administration to a patient.

In the present embodiment, the chambers a, b, c and d are formed between a plurality of partitions 120A, 120B, 120C, 120D, each formed as a movable piston which is sealed against the inner face 122 of housing 110 by a suitable seal, referenced 124. Typically all but the rearmost partition 120D are further formed to include a rupture susceptible portion, shown schematically at 126A, 126B and 126C, which are adapted to rupture in the presence of at least a critical pressure differential thereacross or shearing force applied thereto. In the present embodiment, there is provided a rupture element 128 which is seen, by way of example, to be formed as a hollow spike formed contiguously with outlet port 116. As each partition has a predetermined thickness, and it is desired to completely empty the chamber immediately distal thereto, the rupture susceptible portions are formed to accommodate the hollow spike at positions which require a previous emptying of the immediately preceding chamber.

Driver 118 may be any suitable means for forcing the partitions 120 towards the distal end 112 of the housing. In the present embodiment driver 118 is exemplified as a liquid or gas pressure source, such as an aerosol or other suitable pressurized fluid source, although it could equally be a mechanical driving means, spring-driven or the like, so as to provide a driving force which is sufficient to dispense the pharmacological agents contained in device 100 of the invention, at a predetermined, desired rate.

It is an aim of the present invention to provide a multi-stage drug administration device which is not merely easy to use, but which may also be pre-filled in the factory, with attendant advantages to drugs manufacturers. Accordingly, while different embodiments of the present invention provide different ways to achieve this, as described hereinbelow, in the present embodiment, there is provided a plurality of filler ports 130, spaced along the length of the housing 110, in registration with the chambers A-D, to be filled. Preferably, a plurality of bleeding ports 132 is also provided, so as to enable bleeding of each chamber as it is filled. Clearly, once each chamber is filled, each of the ports 130 and 132 associated therewith is sealed by means of a suitable cap 134.

It will thus be appreciated that the use of device 100 is as follows:

Initially, the partitions 120 are arranged in the positions illustrated in FIG. 1A. Clearly, while the chambers are seen to have similar volumes, the partitions 120 may be arranged, prior to filling, so as to have different volumes, corresponding to different quantities of different pharmacological agents, as may be required.

Once the chambers have been filled and the ports 130 and 132 sealed, an outlet cap 136 (FIG. 1A) is removed, in order to enable outlet port 116 to be connected to an IV port, or the like.

The agent in chamber A is then dispensed by operation of driver 118 which, as mentioned above, is typically a suitable fluid pressure source. Pressurization of the interior of housing 110 between the rearmost partition 120D, closest to proximal end 114, causes the application of a driving force to rearmost partition 120D, and consequently, on the entire array of partitions and the pharmacological agents contained therebetween in chambers A-D, so as to cause the agent contained in chamber a to be dispensed through outlet port 116.

As seen with reference to FIGS. 1A and 1B, as partition 120A approaches distal end 112 of the housing 110, the rupture susceptible portion 126A of partition 120A accommodates almost the entire length of hollow spike 128, until it becomes punctured by the spike 128, as shown in FIG. 1B. At this point, chamber A has been completely emptied, and the agent contained in chamber B begins flowing out through outlet port 116, via spike 128.

It will be appreciated that as the driving pressure is sustained, the contents of the entire device 100 are dispensed as each partition is ruptured by spike 128, and each successive chamber is exhausted, thereby completing the administration of the desired pharmacological agents in a manner which, after initialization, is entirely automatic, and does not require the presence or intervention of medical personnel.

As mentioned above, the basic characteristics of the invention are present in each of the embodiments; each embodiment now being described specifically in respect of its specific features, in conjunction with FIGS. 2-7B, as follows:

Referring now to FIG. 2, there is provided a multi-stage infusion device 200 which is generally similar to device 100 shown and described above in conjunction with FIGS. 1A-1C, except for the provision of a single spike 228 for rupturing partitions 220A-220C during dispensing of the agents contained in the various chambers of the device, as these are driven by driver 218. As seen in the drawings, in the present embodiment, driver 218 is exemplified as a resilient compression member, located within a proximal end portion 214 of housing 210, so as to abut partition 220D.

In the case of the embodiment of FIG. 3, it is seen that the single rupture element of the previous embodiments is replaced by a first rupture element 328 formed within housing 310 adjacent to the outlet port 316, and which is operative to rupture partition 320A upon engagement therewith; and smaller, proximally facing rupture elements 328A and 328B, operative to rupture partitions 320B and 320C when engaged therewith.

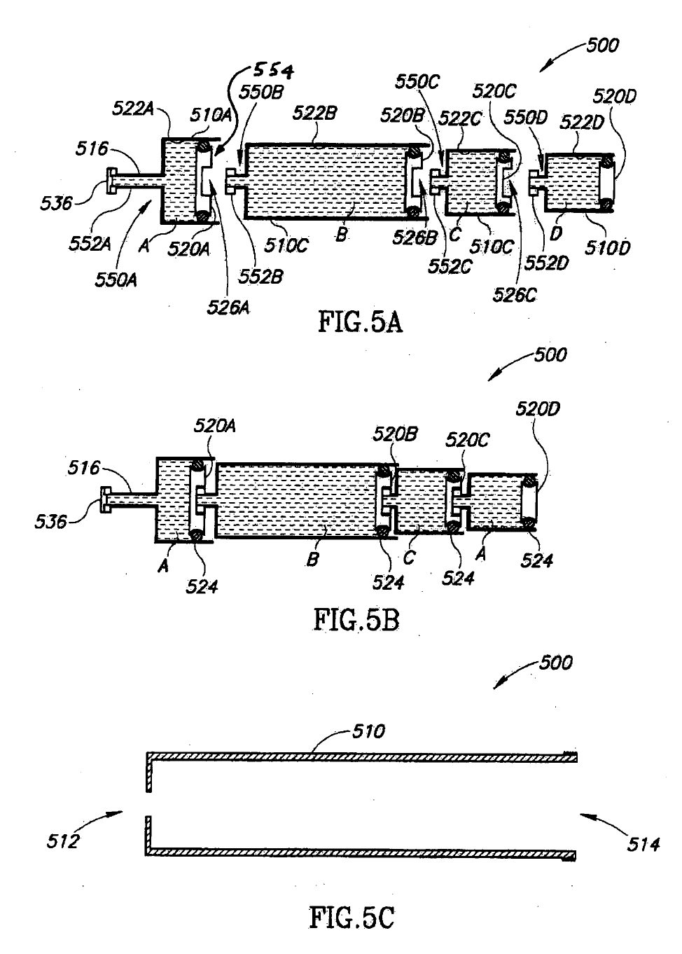

Referring now to FIGS. 4A-4C, it is seen that the housing of device 400 of the present invention may be formed in segments or stages, respectively referenced 410A-410D, which connect together as via screw threads 421 and 423. Each segment includes a single chamber which may be pre-filled with any agent required, and which includes a pair of piston like partitions, generally similar to partitions 120 in the embodiment of FIGS. 1A-1C. The exception to this is distal segment 410A which typically has a single partition 420A only, and which is formed so as to include outlet port 416. In the present embodiment there is also typically provided an additional, proximal end portion 419, associated with driver 418. As seen in the drawings, driver 418 is a pressurized liquid/gas source, operative to drive the partitions and chambers, substantially as shown and described above in conjunction with the embodiment of FIGS. 1A-1C, but with an intervening inflatable drive element 417.

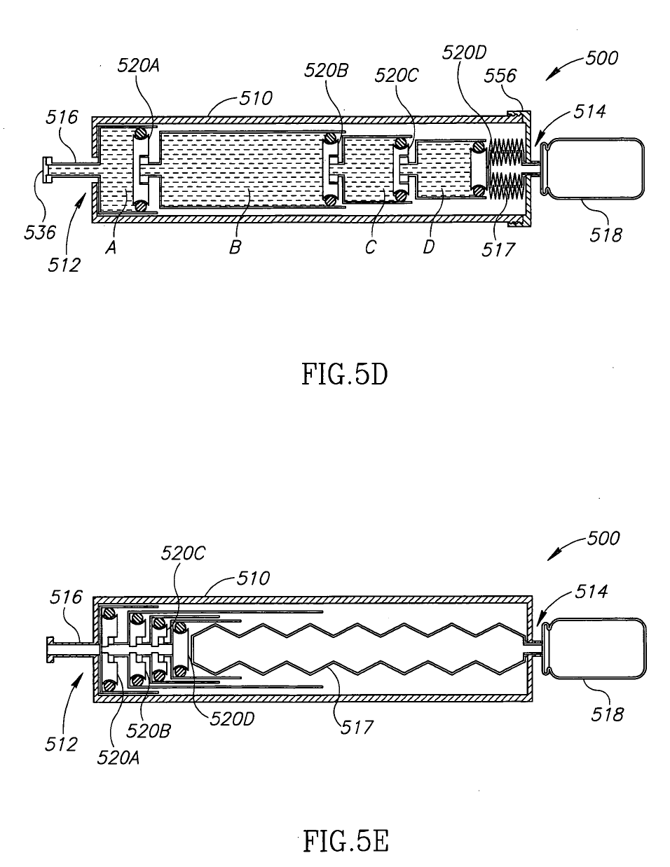

Referring now to FIGS. 5A-5E, in accordance with a further embodiment of the invention, there is provided a multi-stage drug administration device 500, which is also formed with a plurality of segments or stages, as in the embodiment of FIGS. 4A-4C. In the present embodiment however, the different stages are formed so as to be nestable within each other, and so as to have cylindrical housing portions 510A-510D, which nest within each other and which are housed when fully assembled, in housing 510.

As seen, each segment encloses a chamber containing a pharmacological agent as desired, and has a distal end 550 which incorporates an outlet portion 552, and a proximal end 554 in which is disposed a partition 520. Each chamber is filled via outlet portion 552, and is then positioned in abutting engagement with a central, rupture susceptible portion 526 of partition 520. Once assembled, as seen in FIG. 5B, and the assembly is placed within housing 510, an inflatable drive element 517 is then inserted behind and in touching engagement with the proximal partition 520D, as seen in FIG. 5D. Subsequently, housing 510 is closed by an end cap 556, through which a driver 518, exemplified herein as a pressurized liquid/gas source, is placed in operative contact with drive element 517, as seen in FIG. 5D. FIG. 5E illustrates the device 500 after use.

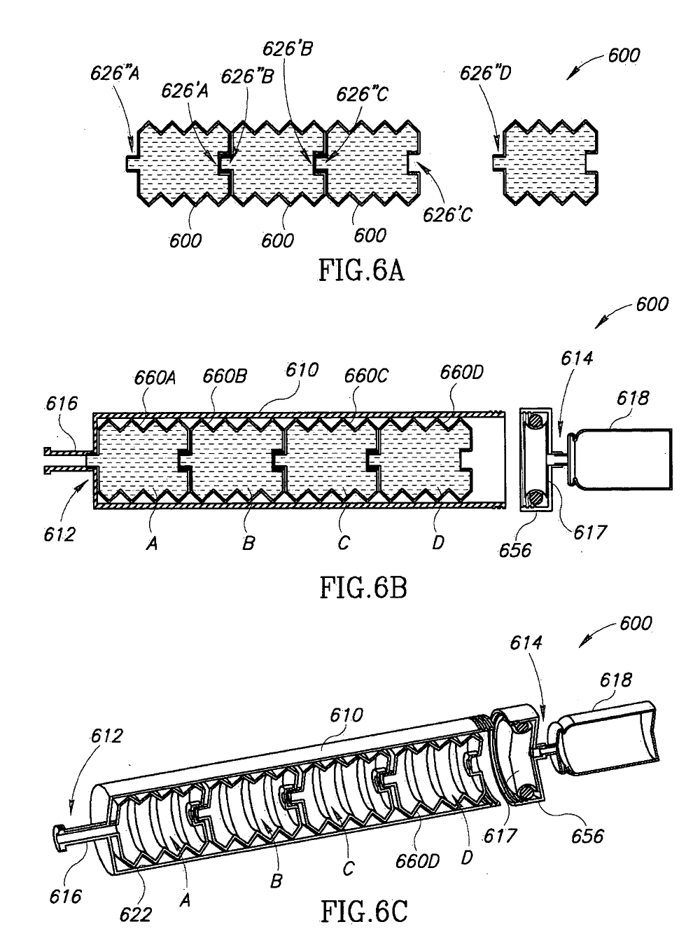

Referring now to FIGS. 6A-6D, there is also provided a multi-stage drug administration device 600 which employs a plurality of pre-filled, pre-sealed, collapsible modules 660. As seen in FIG. 6E, each module 660 is concertina-shaped and has a first rupture susceptible portion 626′ on a proximal side thereof and a second rupture susceptible portion 626″ on a distal side. These rupture susceptible portions are adapted to rupture when subjected to a predetermined driving force, as described hereinabove.

Preferably, the first and second portions 626′ and 626″ are formed as female and male portions, and so as to matingly engage a corresponding portion of an adjacent module, so that an assembly of the modules 660 can be packed together in a desired sequence (FIG. 6A), and then loaded into housing 610. As seen in FIGS. 6B and 6C, a drive element 617, illustrated herein as a piston, is then inserted behind and in touching engagement with the proximal module 660D, and the housing 610 is closed by an end cap 656, through which a driver 618, exemplified herein as a pressurized liquid/gas source, is placed in operative contact with drive element 617. FIG. 6D illustrates the device 600 after use.

Referring once more to FIGS. 6E and 6F, in accordance with one embodiment of the invention, collapsible module 660 may be manufactured in a flat pack configuration, illustrated in FIG. 6D, and so as to be expandable to the configuration seen in FIG. 6F. This is facilitated by the provision of inward facing teeth 670, which are adapted to grip the inward-facing side 672 of first rupture susceptible portion 626′, thereby to substantially prevent any undesired expansion of module 660, prior to use thereof. When it is desired to fill module 660, it may be manually expanded, either before or during filling with a desired pharmacological agent.

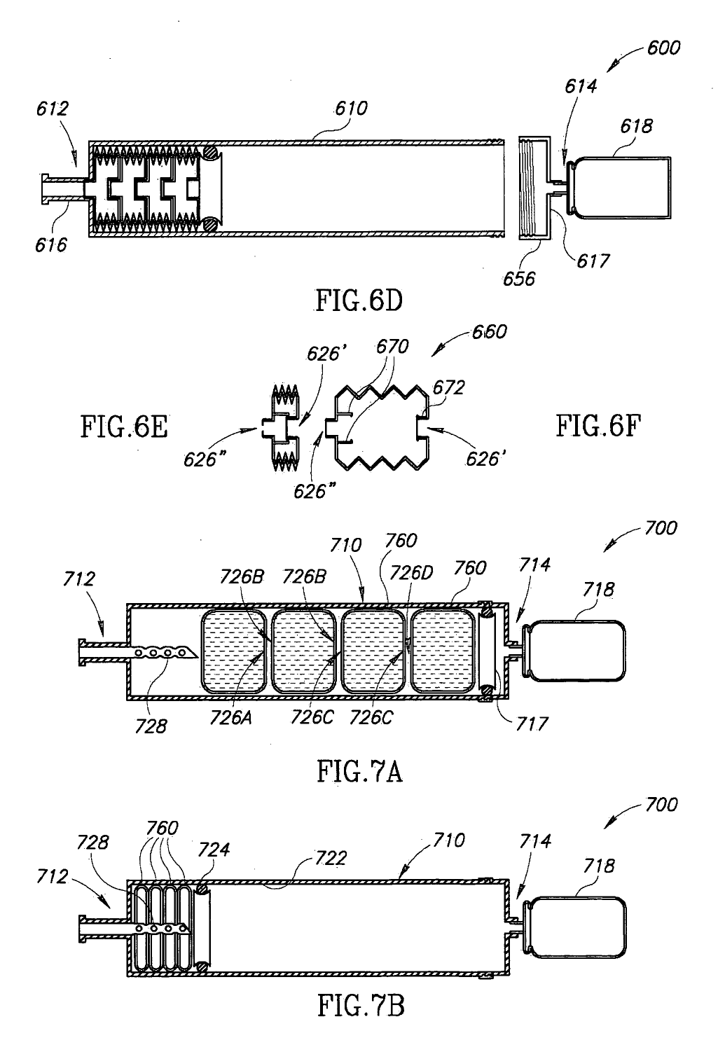

Referring now to FIGS. 7A-7B, device 700 is generally similar to device 600, except that modules 760 are typically smooth walled, preferably being formed of a thin polymer membrane. Furthermore, rupture element 728 is, in the present embodiment, exemplified as a hollow spike, as shown and described above in conjunction with FIGS. 1A-1C. Operation of device 700 by exposure to the pressurized liquid/gas contents of driver 718 is operative to force drive element 717 from its initial, proximal position, shown in FIG. 7A, towards the distal end 712. As this happens, it forces each module in succession into puncturing contact with spike 728, thereby causing the contents of each module to be dispensed successively, in order to provide a user with a predetermined, desired sequence of medication therapy.

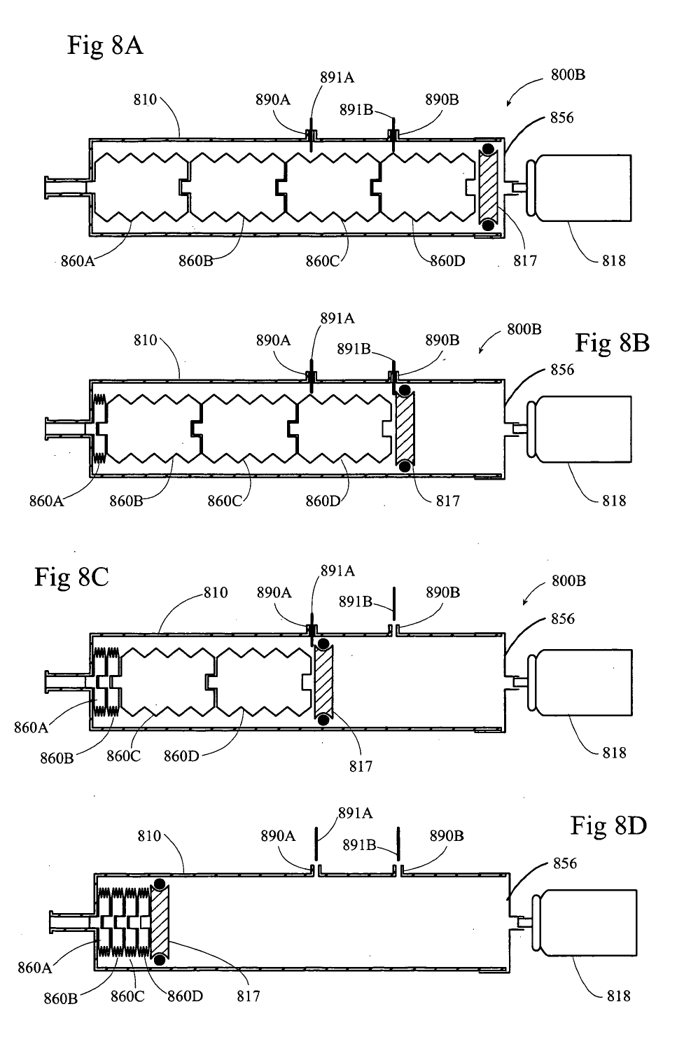

Referring now to FIGS. 8A-8D, there is also provided a multi-stage drug administration device 800 which employs a plurality of pre-filled, pre-sealed, collapsible modules 860. In this preferred embodiment of the invention each module 860 is concertina-shaped and has a first and second rupture susceptible portions on proximal and distal sides thereof, respectively, as in the embodiment shown in FIGS. 6A to 6F, which was described in details hereinabove. Similarly, the first and second rupture susceptible portions may be formed as matingly engageble female and male portions, so that an assembly of the modules 860 can be packed together in a desired sequence, and then loaded into housing 810. A drive element 817, illustrated herein as a piston, is then inserted behind and in touching engagement with the proximal module 860D, and the housing 810 is closed by an end cap 856, through which a driver 818, exemplified herein as a pressurized liquid/gas source, is placed in operative contact with drive element 817.

The drug administration in this preferred embodiment may be halted at predetermined stages of drug administration by stopper pins 891. For this purpose narrow openings 890 are provided on the outer surface of housing 810 such that a portion of said stopper pins 891 may be externally introduced into the interior of housing 810 via said openings 890, such that said opening 890 are sealed by said stopper pins 891. As demonstrated in FIGS. 8A-8D, the drug administration is carried out by the application of pressurized gas/liquid through the end cap 856 via driver 818 attached thereto. If stopper pins 891 are present the advancement of drive element 817 is stopped whenever it reaches the location of a stopper pin 891.

In the example shown in FIG. 8B the drug administration is stopped when drive element 817 reaches the location in which stopper pin 891B is placed such that only the agent in the first module 860A is dispensed. As demonstrated in FIG. 8C, drug administration may continue by removal of stopper pin 891B, and in this example the drug administration will proceed until drive element 817 reaches the location in which stopper pin 891A is placed, such that the agent in the second module 860B is dispensed. Accordingly, the agents in the third and fourth modules, 860C and 860D respectively, may be dispensed by removing stopper pin 891A. FIG. 8D illustrates the device 800 after dispensing all the modules 860.

Collapsible module 860 may be manufactured in a flat pack configuration, similar to that shown in FIG. 6D, which is expandable to the configuration shown in FIG. 6F. Similarly, an inward facing teeth (670 in FIG. 6F) may be utilized to grip an inward-facing side (672 in FIG. 6F) of the first rupture susceptible portion, thereby to substantially prevent any undesired expansion of modules 860, prior to use thereof.

It will be appreciated by persons skilled in the art that the scope of the present invention is not limited by what has been specifically shown and described above in conjunction with the drawings. Rather, the scope of the invention is limited solely by the claims, which follow.

1. A multi-stage drug administration device for administering a sequence of pharmacological agents to a patient, comprising:
a generally cylindrical housing having distal and proximal ends, having formed at said distal end an outlet port for connection to an IV port; and
at least first and second compressible, mutually sealed chambers located within said housing between said distal and proximal ends, for containing therebetween in mutual isolation a corresponding plurality of pharmacological agents for sequential administration to a patient, wherein a rupture susceptible portion of a common dividing wall between adjacent chambers is adapted to rupture in the presence of at least a critical pressure differential thereacross,
wherein upon exposure to a predetermined source of pressure associated with said proximal end of said housing, said plurality of sealed chambers becomes compressed towards said distal end so as to cause an outflow of the pharmacological agent in said first chamber through said outlet port,
and wherein, at the time of emptying of said first chamber, the pressure differential across said rupture susceptible portion of said common dividing wall reaches said critical pressure differential causing rupture thereof, and a consequent outflow of the pharmacological agent contained in said second chamber through said outlet port.

2. The device of claim 1, wherein said common dividing wall is a disk-shaped piston spaced apart along the length of the cylinder, each said piston being circumferentially sealed with an interior wall surface of said cylinder, thereby to form said plurality of chambers, each said piston being adapted to rupture in the presence of said critical pressure differential.

3. The device of claim 1, further comprising puncturing means located at said distal end, for puncturing a piston proximal thereto, in the presence of said critical pressure differential.

4. The device of claim 3, wherein each said piston has formed thereon additional puncturing means for puncturing an adjacent piston when brought into proximity therewith.

5. The device of claim 1, wherein each said chamber includes a volume of a preselected pharmacological agent, contained within an impervious membrane.

6. The device of claim 1, wherein said impervious membrane is a flexible membrane.

7. A multi-stage drug administration system for administering a sequence of pharmacological agents to a patient, comprising:
a generally cylindrical housing having distal and proximal ends, having formed at said distal end an outlet port for connection to an IV port;
at least first and second compressible, mutually sealed chambers located within said housing between said distal and proximal ends, for containing therebetween in mutual isolation a corresponding plurality of pharmacological agents for sequential administration to a patient, wherein a rupture susceptible portion of a common dividing wall between adjacent chambers is adapted to rupture in the presence of at least a critical pressure differential thereacross; and
a source of pressure associated with said proximal end of said housing,
wherein upon exposure to said pressure source, said plurality of sealed chambers becomes compressed towards said distal end so as to cause an outflow of the pharmacological agent in said first chamber through said outlet port,
and wherein, at the time of emptying of said first chamber, the pressure differential across said rupture susceptible portion of said common dividing wall reaches said critical pressure differential causing rupture thereof, and a consequent outflow of the pharmacological agent contained in said second chamber through said outlet port.

8. A multi-stage drug administration system for administering a sequence of pharmacological agents to a patient, comprising:
a plurality of mutually connectable sealed modules, each containing a preselected pharmacological agent, arranged in a connected stack which includes a proximal end module and a distal end module;
a dispensing module connected to said distal end module; and
a pressure module, for connecting a source of pressure to said proximal end module.

9. The system of claim 8, further comprising a housing and a drive element, wherein said housing is adapted for disposing the modules and said drive element thereinside such that said drive element is in contact with the proximal module, and for attaching the dispensing module to a distal end thereof and the pressure module to a proximal end thereof.

10. The system of claim 9, wherein said housing comprises one or more narrow openings through which portions of stopper pins may be externally introduced into the interior of the housing such that drug administration is halted whenever the drive element reaches the location of a stopper pin which blocks further advancement thereof.

11. A method of administering a sequence of pharmacological agents to a patient, comprising:
providing a multi-stage drug administration device comprising:
a generally cylindrical housing having distal and proximal ends, having formed at said distal end an outlet port for connection to an IV port; and
at least first and second compressible, mutually sealed chambers located within said housing between said distal and proximal ends, for containing therebetween in mutual isolation a corresponding plurality of pharmacological agents for sequential administration to a patient, wherein a rupture susceptible portion of a common dividing wall between adjacent chambers is adapted to rupture in the presence of at least a critical pressure differential thereacross,
and
exposing said device to a predetermined source of pressure associated with said proximal end of said housing,
such that said plurality of sealed chambers becomes compressed towards said distal end so as to cause an outflow of the pharmacological agent in said first chamber through said outlet port,
and such that, at the time of emptying of said first chamber, the pressure differential across said rupture susceptible portion of said common dividing wall reaches said critical pressure differential causing rupture thereof, and a consequent outflow of the pharmacological agent contained in said second chamber through said outlet port.

12. The method of claim 11 wherein said common dividing wall of said device is a disk-shaped piston spaced apart along the length of the cylinder, each said piston being circumferentially sealed with an interior wall surface of said cylinder, thereby to form said plurality of chambers, each said piston being adapted to rupture in the presence of said critical pressure differential.

13. The method of claim 11 wherein said device further comprises puncturing means located at said distal end, for puncturing a piston proximal thereto, in the presence of said critical pressure differential.

14. The method of claim 13 wherein each said piston has formed thereon additional puncturing means for puncturing an adjacent piston when brought into proximity therewith.

15. The method of claim 11 wherein each said chamber includes a volume of a preselected pharmacological agent, contained within an impervious membrane.

16. The method of claim 11 wherein said impervious membrane is a flexible membrane.

17. A method of administering a sequence of pharmacological agents to a patient, comprising:
providing a multi-stage drug administration system comprising:
a plurality of mutually connectable sealed modules, each containing a preselected pharmacological agent, arranged in a connected stack which includes a proximal end module and a distal end module;
a dispensing module connected to said distal end module; and
a pressure module, for connecting a source of pressure to said proximal end module;
and
exposing said system to said pressure module,
such that said plurality of sealed modules becomes compressed towards said distal end module so as to cause an outflow of the pharmacological agent in a respective one of said sealed modules through said dispensing module.

18. The method of claim 17, wherein said system further comprises a housing and a drive element, wherein said housing is adapted for disposing the modules and said drive element thereinside such that said drive element is in contact with the proximal module, and for attaching the dispensing module to a distal end thereof and the pressure module to a proximal end thereof.

19. The method of claim 18, wherein said housing comprises one or more narrow openings through which portions of stopper pins may be externally introduced into the interior of the housing such that drug administration is halted whenever the drive element reaches the location of a stopper pin which blocks further advancement thereof.
